(19) United States
(12) Patent Application Publication
(10) Pub. No.: US 2024/0327641 A1
(43) Pub. Date: Oct. 3, 2024

(54) BIODEGRADABLE MOLDABLE SOY-BASED FOAMS

(71) Applicant: Indiana Soybean Alliance, Inc., Indianapolis, IN (US)

(72) Inventors: Louis Edwards Caceres-Martinez, Lafayette, IN (US); Valeria Briceida Tellez-Gallego, Lafayette, IN (US); Amy Tang, São Paulo (BR); Alyssa Lynn Choi, Addison, IL (US)

(21) Appl. No.: 18/621,012

(22) Filed: Mar. 28, 2024

Related U.S. Application Data
(63) Continuation of application No. 63/492,722, filed on Mar. 28, 2023.

(51) Int. Cl. C08L 89/00; C08K 5/00; C08K 5/053; C08K 5/092; C12P 1/02

(52) U.S. Cl. CPC C08L 89/00; C08K 5/0008; C08K 5/053; C08K 5/092; C12P 1/02; C08K 2201/014; C08L 2201/06; C08L 2203/14; C08L 2205/03; C08L 2205/06; C08L 2312/00

(57) ABSTRACT

Aspects of the invention include environmentally friendly plastic-free alternatives to polystyrene and methods of making the same. The inventive material is a biodegradable, compostable and non-toxic soy-based foam that can be used to produce different density packaging materials/cushions/foams for protecting valuable merchandise, such as electronics, housewares and other consumer items during shipping to customers.

### PRIORITY CLAIM

This application claims the benefit of U.S. Provisional No. 63/492,722, filed on Mar. 28, 2023, and incorporated herein by reference in its entirety.

### TECHNICAL FIELD

Aspects of the invention related to biodegradable moldable foams comprised in part of proteins derived from plants, including proteins isolated from soybean plants.

### BACKGROUND

Commercially available moldable foams are widely used to create a wide variety of products including materials used in construction and packaging. Many of these foams such as polystyrene are petroleum based and have long resonance time in the environment. Many of these foams degrade into particles of microplastics, which are slow to degrade, can find their way into living organisms and further damage the environment. Accordingly, there is a need for more ecologically friendly moldable foams for use in applications such as building materials and packaging materials.

### SUMMARY

Some embodiments of the invention include soy-based moldable foam comprising cross-linked soy proteins and gas bubbles entrapped with the cross-linked soy-protein matrix and obtained by degradation of hydrogen peroxide catalyzed with active dry yeast. Some inventive foams include glycerin (for example, glycerin derived from soybeans) soy wax, bases, salts, components of cross-linking agents such as dicarboxylic acids, and/or surface-active agents such as soaps and/or surfactants.

Some molded foams may have densities in the range of about 70 kg/cm³ to about 350 kg/cm³. The density of the foam can be tuned for varying the conditions under which the process of making is conducted or by varying the types and amounts of compounds used to make the cross-linked matrix and/or the bubble added to the matrix to form the foam. It should be possible to create foams that are denser or less dense than the range of densities exemplified herein.

Still other embodiments include a method of making soy-based moldable foams. Some of these methods include: mixing at least one compound rich in soy-based proteins; homogenizing the mixture, adding at least one agent that helps to crosslink at least some of the proteins in soy-based substantially homogenous mixture to create a soy-based protein cross-linked matrix; providing one or more sources of gaseous bubbles, for example, agents that form bubbles, optionally activating the bubble producing mechanism or chemical agent(s) to form a foaming solution; forming a foam formulation by adding the foaming mixture to the cross-linked soy based protein mixture, optionally adding the foam formulation to one or more molds to form a molded foam; and drying the molded foam.

In some embodiments, the inventive soy-based foam can be dried without adding it to a mold. Foam allowed to dry without first being molded can create an article of foam that can be used, for example, as loose packing material.

In some embodiments, the crosslinking agents may be one or more dicarboxylic acids such as maleic acid, succinic acid, glutaric acid and the like. In some embodiments, crosslinking agents other than dicarboxylic acids may be used.

In some embodiments, the foaming mixture is formed by adding a source of gas bubbles to the cross-linked foam. In some embodiments, the source of bubbles can be hydrogen peroxide in reaction with active dry yeast. In still other embodiments, the source of bubbles can be other inorganic compounds such as sodium bicarbonate.

One embodiment of the invention is formed by a mixture of soy protein, soy wax, soy glycerol, maleic acid (which promotes soy protein cross-linking), sodium hydroxide, dish soap which acts as surfactant, table sugar and active dry yeast. See, for example, FIG. 1.

Embodiment 1: A biodegradable foam, including:
a cross-linked protein;
a gas;
inactivated yeast; and a meltable wax;
wherein, the protein forms a matrix that includes one or more spaces that entraps the gas and the matrix interacts with the wax.

Embodiment 2: The foam according to embodiment 1, further including a plasticizer.

Embodiment 3: The foam according to embodiment 1, wherein the protein is selected from the sources consisting of: soybeans, peas hemp, milk, flaxseed, whey, rice powder, zein, egg whites, fish, bacteria, yeast, and fungi.

Embodiment 4: The foam according to embodiment 1, wherein the gas is selected from the group consisting of: carbon dioxide, oxygen, nitrogen, an inert gas, and air.

Embodiment 5: The foam according to embodiment 1, wherein the foam further includes an excipient.

Embodiment 6: The foam according to embodiment 5, wherein the excipient is selected from the group consisting of: sugar, polyvinylpyrrolidone, a fiber, and at least one emulsifier.

Embodiment 7: The foam according to embodiment 6, wherein the emulsifier is selected from the group consisting of: soap and lecithin.

Embodiment 8: The foam according to embodiment 1, wherein the protein is isolated from soy.

Embodiment 9: The foam according to embodiment 4, wherein the gas is carbon dioxide.

Embodiment 10: The foam according to embodiment 4, wherein the gas is oxygen, and the oxygen is provided by a reaction between yeast and hydrogen peroxide during the process of forming the foam.

Embodiment 11: The foam according to embodiment 6, wherein the excipient is sugar.

Embodiment 12: The foam according to embodiment 6, wherein the excipient is polyvinylpyrrolidone.

Embodiment 13: The foam according to embodiment 1, wherein the meltable wax melts over a temperature range selected from the group consisting of: about 50° C. to about 150° C.; about 70° C. to about 120° C.’ and about 80° C. to about 100° C.

Embodiment 14: The foam according to embodiment 13, wherein the wax is selected from the group consisting of: soy wax, paraffin, beeswax, candelilla wax, coconut wax, casting wax, microcrystalline wax, carnauba.

Embodiment 15: The foam according to embodiment 2, wherein the plasticizer is glycerin.

Embodiment 16: The foam according to embodiment 6, wherein the excipient is a fiber.

Embodiment 17: The foam according to embodiment 16, wherein the fiber is selected from the group consisting of: soy fiber, corn stalk fiber, and hemp fiber.

Embodiment 18: The foam according to embodiment 1, wherein the cross-linked protein includes a crosslinker.

Embodiment 19: The foam according to embodiment 18, wherein the cross linker is a dicarboxylic acid selected from the group consisting of: malic acid, aspartic acid, adipic acid dihydrazide, adipic acid, glutamic acid, citric acid, tartaric acid, oxalic acid, azelaic acid, fumaric acid.

Embodiment 20: The foam according to embodiment 1, further including a salt.

Embodiment 21: The foam according to embodiment 20, wherein the salt is selected from the group consisting of sodium carbonate; sodium malate; and potassium bicarbonate.

Embodiment 22: The foam according to embodiment 1, wherein the foam, includes: about 6 to 10% wt./wt. protein;
about 0.3 to 0.4% wt./wt. of dicarboxylic acid;
about 1.0 to 1.3% wt./wt. of a strong base;
about 3 to 7% wt./wt. glycerol;
about 3 to 7% wt./wt. of a wax;
about 1.5 to 10% wt./wt. of hydrogen peroxide;
about 4.5 to 6.5% wt./wt. of soap; and about 5 to 7% wt./wt. of yeast.

Embodiment 23: The foam according to embodiment 22, wherein the foam, further includes:
about 1 to 5% Soy fiber;
about 2 to 10% soy flour; and about 0.2 to 2% PVP.

Embodiment 24: The foam according to embodiment 1, wherein the foam has a density of between about 60 kg/m3 to about 400 kg/m3.

Embodiment 25: The foam according to embodiment 1, wherein the foam has a density of between about 70 kg/m3 to about 350 kg/m3.

Embodiment 26: The foam according to embodiment 1, wherein the foam has a density of between about 78 kg/m3 to about 335 kg/m3.

Embodiment 27: A method of making a biodegradable foam, including:
a. adding, with agitation, a crosslinking reagent to an aqueous suspension of a protein held at a temperature of between 80° C. and 100° C. form a mixture;
b. introducing, with stirring, a plasticizer into the mixture;
c. supplying the mixture with a melted meltable wax to create a formulation;
d. providing a source of a gas to the formulation to form a liquid foam; and
e. drying the liquid foam to form a stable foam.

Embodiment 28: The method according to embodiment 27, further including: emulsifying the formation to create an emulsion before providing the gas to the emulsion.

Embodiment 29: The method according to embodiment 27, wherein the source of gas is provided by adding an activated yeast suspended in water; and a solution of hydrogen peroxide, corn syrup and soap to the emulsion.

Embodiment 30: The method according to embodiment 29, wherein the amount of active yeast added is about 26% wt./wt.; the amount of hydrogen peroxide added is about 30% wt./wt., the amount of soap is about 6 to about 25% wt./wt. and the amount of corn syrup is about 6% wt./wt. of the source of the gas.

Embodiment 31: The method according to embodiment 29, wherein the activated yeast suspension and the solution of hydrogen peroxide, corn syrup and soap are prepared separately and then added to the emulsion at about the same time.

Embodiment 32: The method according to embodiment 27, wherein the protein is recovered form a source selected from the group consisting of: a plant, an animal, a bacterium, a yeast and a fungus.

Embodiment 33: The method according to embodiment 27, wherein the protein is selected from the group consisting of: soybeans, flaxseed, whey, rice powder, zein, egg white, casein and fish.

Embodiment 34: The method according to embodiment 27, wherein the mixture includes a flour.

Embodiment 35: The method according to embodiment 34, wherein the flour is a soy flour.

Embodiment 36: The method according to embodiment 27, wherein the protein is a soy isolate.

Embodiment 37: The method according to embodiment 27, wherein the crosslinking reagent includes a dicarboxylic acid and a base.

Embodiment 38: The method according to embodiment 37, wherein the dicarboxylic acid: is selected from the group consisting of: malic acid, aspartic acid, adipic acid dihydrazide, adipic acid, glutamic acid, citric acid, tartaric acid, and oxalic acid.

Embodiment 39: The method according to embodiment 37, wherein the base selected from the group consisting of: sodium hydroxide and calcium hydroxide.

Embodiment 40: The foam according to embodiment 27, wherein the formulation includes:
about 1:10 to 1:5 wt./wt. protein;
about 1:44 wt./wt. of a mix including a dicarboxylic acid and a strong base;
about 2:45 to 1:11 wt./wt. glycerol; and about 2:33 to 5:2 wt./wt. wax.

Embodiment 41: The foam according to embodiment 27, wherein the source of the gas includes:
about 8 to 20% of a 30% solution of hydrogen peroxide and water;
about 6 to 24% soap;
about 3 to 10% corn syrup; and about 20 to 30% yeast.

Embodiment 42: The method according to embodiment 27, furthering including transferring the liquid foam to a mold; and drying the liquid foam to form a dried foam.

Embodiment 43: The method according to embodiment 42, furthering including the step of shaping the dried foam.

### BRIEF DESCRIPTION OF THE DRAWINGS

FIG. 1. is a graphical representation of one inventive foam composition;

FIG. 2. is a flow chart showing some of steps in the process of producing one of the inventive foams;

FIG. 3. is a cartoon showing soy-protein crosslinking on the molecular level using, in this example, the dicarboxylic acid maleic acid; and

FIG. 4. is a cartoon showing a bench level method of producing bubbles to add to the cross-linked soy protein to form a foam.

### DESCRIPTION

Disclosed herein is an environmentally friendly soy-based foam that is a plastic-free alternative to polystyrene and other material used in applications such as packing and construction. The soy-based foam is a biodegradable, compostable, and non-toxic soy-based foam, which can be used to produce different density packaging materials/cushions/foams for protecting valuable merchandise, such as electronics, houseware and other consumer items during shipping to customers.

### Definitions

Unless explicitly stated otherwise or clearly implied otherwise the term “about” refers to a set of values of plus or minus 10 percent, for example, about 1.0 encompasses values of 0.9 to 1.1.

Crosslinked proteins are proteins that are linked to themselves or to one or more other distinct proteins. For example, different elements of the same polypeptide can be crosslinked by crosslinks between different elements of the same polypeptide. In another example, two or more distinct polypeptides may be crosslinked to one another. In still another example, crosslinked proteins may include crosslinks between both elements of the same polypeptide and one or more distinct polypeptides. Crosslinked proteins may include portions of crosslinking agents present in the cross-link. For example, proteins crosslinked by treatment with dicarboxylic acids may include element of the acid in the crosslink.

Meltable waxes, include waxes that can transition from a malleable solid or semi-solid to a liquid without decomposing. Temperatures for such transitions for such waxes, include but are not limited to temperatures, in one or more of the following ranges of about: 50° C. to 160° C.; 60° C. to 120° C.; and 80° to 100° C.

The term ‘plasticizer’ refers to a substance added to matrix to produce and/or promote plasticity and/or flexibility and/or reduce brittleness. One such plasticizer that can be used to practice some aspects of the invention is glycerin.

Proteins that can be used to practice at least some aspects of the invention, include, but are not limited, to proteins recovered and/or isolated from plants such as: soybean, peas, hemp, potato, wheat, corn, mullet, rice, and the like. Proteins from other sources such as bacteria, yeast, fungi, and/or animals can also be used to practice some elements of the invention. Preferable, the proteins used in the inventive foams are generally regarded as biodegradable.

Waxes that can be used to practice at least some aspects of the invention, include, but are not limited to; soy wax, bees wax, carnauba wax, paraffin, candelilla wax, casting wax.

Crosslinking agents that can be used to create crosslinked proteins include, but are not limited, chemical that have two or more groups that react with elements, such as specific amino acids, in polypeptide chains. For example, dicarboxylic acids such as, and the like can be used to crosslink proteins in some aspects of the invention. Preferably, the crosslinking agents or at least the portions of the crosslinking agent that form the crosslinks are biodegradable.

Plasticizers that can be used to practice some aspects of the invention include, for example, compounds such as glycerol, sources of glycerin include but are not limited to glycerin recovered and/or isolated from natural sources such as fats or triglycerides from plants, animals and fungi, or from naturally occurring or bioengineered microbes such as bacteria, yeasts, and fungi. Glycerin from synthetic or semi-synthetic sources can also be used to practice some aspects of the invention.

Other plasticizers that can be used to practice the invention, include, but are not limited to; glycerol esters, polyethylene glycols, propylene glycols, sorbitol, epoxidized soybean oil and the like.

This eco-friendly soy-based foam can replace polystyrene for packaging (and insulation) purposes. Various embodiments of the invention not only offer equivalent commercial performance versatility to polystyrene, but are also economically competitive with the petroleum-based, wood-based, building and packaging materials. Replacing petroleum-based products is critical in the packaging industry given the unsustainability and catastrophic impacts on the environment and human health associated with the use of polystyrene.

Accordingly, the inventive foam has the potential to reduce and even eliminate Styrofoam final disposal, which currently encompasses 30% of landfills in the United States and does not decompose, creating a vast environmental impact on land and marine life. The annual estimated cost to landfill polystyrene is calculated to be in the order of about $665 million per year. Switching to a biodegradable alternative has the potential to save millions of dollars in disposal costs alone.

Much like materials such as polystyrene, the inventive soy-based foam can be molded into a variety of shapes and can therefore be used in a wide variety of applications. The soy-based foam materials can be used as a packaging material/cushion/foam to protect valuable merchandise while shipped to customers. These foams can be used, for example, to protect electronics from vibration, abrasion, and humidity, while providing cushioning and an anti-static effect. Moreover, this soy-based packaging material obeys sustainable and environmental principles. It is not only biodegradable but also a compostable alternative to conventional polystyrene packaging materials.

The addition of a melted wax, to a mixture comprising an aqueous suspension of protein and an acid base mixture including an acidic crosslinking reagent and a base dramatically increases the mixture ability to form a stable foam when the mixture is provided with a source of a gas. Combinations of reagents that produce gas in situ appear to be especially efficient sources of the gas required to create a foam. The addition of a plasticizer to the mixture at or before the addition of the wax helped to produce a stable, moldable foam that is more resistant to cracking than similar compositions made without the addition of the plasticizer.

Yeast reacts with hydrogen peroxide to form oxygen and water. All of the products and residual components of this reaction, water, oxygen and inactive yeast found in the final molded foam are safe for the environment while other method of adding a gas to the mixture of crosslinked matrix are available, the combination of yeast and hydrogen peroxide provide a convenient and environmentally safe method for inflating the foam.

Certain preferred embodiments include, a biodegradable foam composed of about 27% wt./wt. cross-linked soybean protein in combination with 0.2% wt./wt. maleic acid and 0.6% wt./wt. sodium hydroxide with 20% of glycerin and 20% wt./wt. soybean wax wherein, the protein forms a matrix that includes one or more spaces that entraps the gas and the matrix interact with the wax. Such gas created by the catalitic activity of yeast in the degradation of hydrogen peroxide to water and oxygen in the presence of 3% wt./wt. regular sugar and 2.4% wt./wt. soap. Such foam is formed by mixing F08-1A, F08-1B, or E02 and FM1-1A as described herein. Specifically, F08-1A, F08-1B, or E02 as the base emulsion, FM1-1A-agent 1 acts as the catalyst, and FM1-1A-agent 2 serves as the substrate for the catalytic reaction. The resultant foam is shaped, molded, and subsequently dried in a convection oven set at 40° C., for 3 to 6 hours, or overnight, rendering it suitable for use in desired packaging and insulation applications.

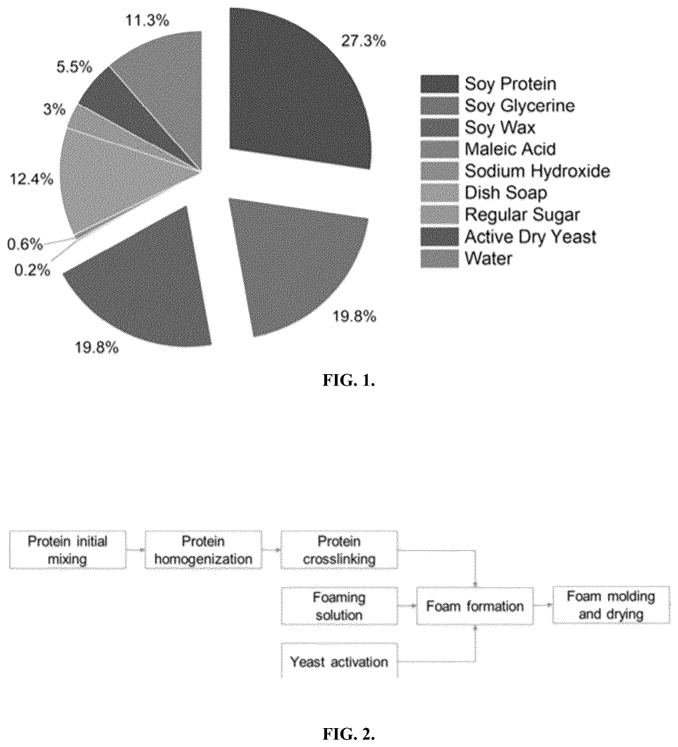

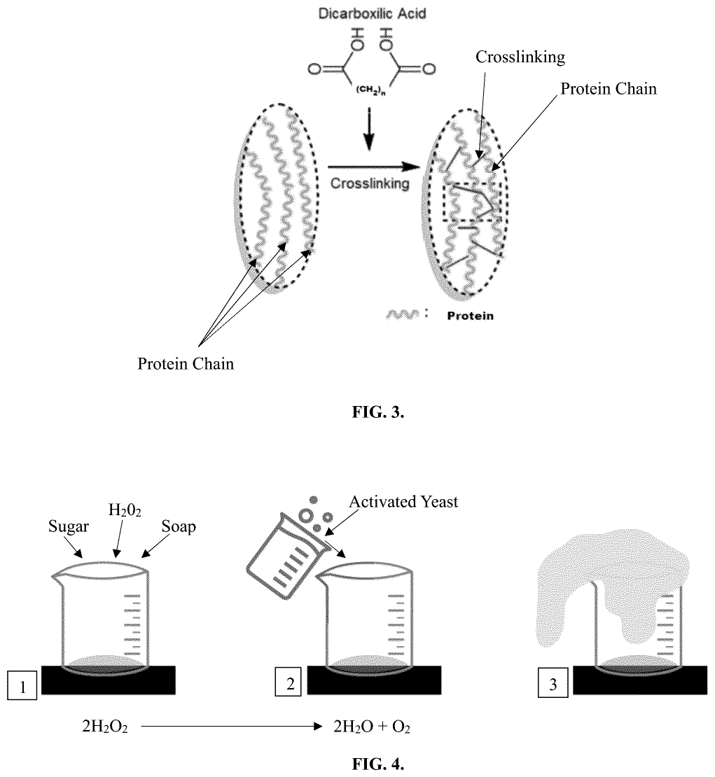

Referring now to FIG. 1, this graph summarizes the elements of one non-limiting embodiment of the invention, specifically; 27.3% soy protein, 19.8% soy glycerine, 19.8% soy wax, 0.2% maleic acid, 0.6% sodium hydroxide, 12.4% dish soap, 3% sugar, 5.5% active dry yeast and 11.3% water.

Those skilled in the art will appreciate that various combination of the disclosed components in various ratios disclosed in the examples disclosed herein as well as materials and methods suggested by these examples can be adjusted to create moldable foams of various desired shapes. And the drying conditions as disclosed herein can be configured to help to yield moldable biodegradable foam compositions.

The disclosed biodegradable foams, as described herein, are particularly well suited for packaging and insulation purposes. The foams exhibit excellent flexibility and mechanical strength, making them ideal for molding into various shapes and configurations to accommodate different packaging needs. Thus, the foams can be effectively employed in packaging applications to protect and cushion packaged items as blocks, sheets, custom shapes, molded inserts, and peanuts.

Some aspects of the invention may use an emulsion for manufacturing a biodegradable foam, the emulsion comprising on a wt./wt. basis: protein in the range of about 1:10 to about 2:10; an acid base solution comprising, a dicarboxylic acid and a base, in the range of about 1:44 to about 2:44; a plasticizer, such as glycerin, in the range of about 2:45 to about 1:11; and a meltable wax in the range of about 2:33 to 5:2 wt./wt. of the emulsion.

Some aspects of the invention may use an emulsion for manufacturing a biodegradable foam, the emulsion comprising on a wt./wt. basis: protein in the range of about 1:20 to about 1:10; an acid base solution comprising, a dicarboxylic acid and a base, in the range of about 1:88 to about 4:44; a plasticizer, such as glycerin, in the range of about 2:90 to about 2:1; and a meltable wax in the range of about 1:33 to 10:2 wt./wt. of the emulsion.

Some aspects of the invention may use an in situ source of gas for manufacturing a biodegradable foam, the in situ source of gas comprising on a percentage basis (%): about 3 to about 70% of a 15 percent solution of hydrogen peroxide; about 6 to about 24% soap or another emulsifier such as lecithin; about 2 to 10% corn syrup; and about 25 to about 40% yeast.

Some aspects of the invention may use an in situ source of gas for manufacturing a biodegradable foam, the in situ source of gas comprising on a percentage basis (%): about 30 to about 70% of a 30 percent solution of hydrogen peroxide; about 10 to about 16% soap or another emulsifier such as lecithin; about 4 to 8% corn syrup; and about 30 to about 35% yeast

Some non-limiting exemplary parameters for manufacturing some foams according to some aspects of the invention include creating and holding a suspension comprising protein and water a temperature of between about 50° C. and 100° C., and mixing the same for between about 30 minutes to about 2 hours; adding a melted wax to the protein suspension at a temperature of between about 60° C. to about 150° C., activating a yeast at a temperature between about 21° C. to about 40° C. for between about 5 minutes to about 25 minutes; drying a liquid foam at a temperature of between about 25° C. to about 60° C. for between about 3 hours to about 12 hours.

Some non-limiting exemplary parameters for manufacturing some foams according to some aspects of the invention include creating and holding a suspending comprising protein and water a temperature of between about 50° C. and 100° C., and mixing the same for between 30 minutes and about 2 hours; adding a melted wax to the protein suspension at a temperature of between about 60° C. to about 120° C., activating a yeast at about 40° C. for between about 5 to about 25 minutes; adding the activated yeast to the protein mixture at about 40° C.; adding the foaming solution at about 40° C.; and drying a liquid foam at a temperature of between about 25° C. to about 60° C., for between about 3 hours to about 12 hours.

### EXAMPLES

All examples and experimental data, whether actual or prophetic, are non-limiting. All examples and data presented herein are presented by way of explanation and not limitation.

We have determined that parameters such as the amount of soy protein in a given formulation, the degree of crosslinking between soy proteins in the foam, mixing conditions (temperature, speed, rate of water loss, and the like), the order of the addition of the components to form the foam and the drying conditions used to create the final molded articles of manufacture can be fine-tuned to create products that have desirable physical characteristics.

### Example 1

Referring now to FIG. 1. A graphical representation of the composition of one embodiment of the invention. In this non-limiting example, the inventive foam is comprised of about 67% wt./wt. of soy derivatives. This exemplary foam is comprised of soy protein, soy wax and excipients that help to maintain a robust structure and shape of the foam after the drying step. In this embodiment, soy glycerin is included to help to maintain foam flexibility.

In still other embodiments of the invention, greater percentages of soy (around 80% wt./wt.) can be achieved by formulating the foams to include emulsifiers such as lecithin to replace the soap. These formulations may exhibit beneficial additional antistatic properties. In some embodiments, the formulation may include polyvinylpyrrolidone, which may be acting as an excipient helping to maintain bubbles in the system and to tune the general degradability of the product while not inducing microplastic formation.

### Example 2

One method of fabricating one or more of the inventive foams disclosed herein comprises a series of steps as summarized in FIG. 2. Briefly, the process can be represented as a series of steps including mixing the protein, homogenizing the protein mixture, and crosslinking at least some of the proteins in the homogenous protein mixture, adding a plasticizer, a wax to form a crosslinked protein mixture with good mechanical properties. Creating a foaming solution and adding the foaming solution to the cross-linked protein mixture to help initiate foam formation and solidification. In some embodiments, at this stage in the process, yeast is activated as part of the foam forming step. Next, the foam may be added to a mold and then dried to form the final molded foam product.

In one embodiment, a protein-acid-water solution is prepared and heated to about 80° C. Concurrently, a foaming solution is prepared. The foaming solution is then mixed with protein-acid-water solution once water has been evaporated. The wet-foam product may be poured into a mold and dried in a convection oven at about 40° C.

### Protein Crosslinking Agents and Formulations

### Examples 1&2

Still other exemplary methods of manufacturing the inventive foams and molded products include the following steps. First mixing the soy proteins, homogenizing the soy protein mixture, treating the homogenized soy-protein mixture with at least one cross-linking agent; providing a source of gaseous bubbles to add to the soy-protein mixture; adding the gaseous bubbles to the mixture to form a foam, optionally adding a compound that catalyzes bubble formation; forming a soy-based foam and optionally adding the foam to a mold and then drying the mold to form an final article of manufacture.

### Example 3

F08-A and F08-B A 20% wt./wt. aqueous solution of maleic acid solution and a 20% wt./wt. aqueous solution of sodium hydroxide are prepared and mixed in a 1:3 wt./wt. ratio referred to as acid base solution (ABS). A suspension comprising 10% wt./wt. soy protein isolate is prepared and sonicated for three minutes. This suspension is made twice for testing two different trials. The first trial is for use as reference (F08-A). The second trial (F08-B), comprises 13% wt./wt. of epoxidized soybean oil with respect to the original solution is added. Half of both these solutions are neutralized and films are obtained.

### Example 4

F08-C A suspension comprising 10% wt./wt. soy protein isolate is prepared and sonicated for three minutes. A 20% wt./wt. aqueous solution of maleic acid solution and a 20% wt./wt. aqueous solution of sodium hydroxide is prepared and mixed in a 1:3 wt./wt. ratio referred to as acid base solution (ABS). The ABS is slowly added to the soy protein suspension while stirring at 80-100° C. in a boiling water bath. A ratio of 1:2, wt./wt. soy glycerin to protein is added slowly to the previous mixture. A film is obtained with good flexibility.

### Example 5

F08-D A suspension comprising 10% wt./wt. soy protein isolate is prepared and sonicated for three minutes. A 20% wt./wt. aqueous solution of maleic acid solution and a 20% wt./wt. aqueous solution of sodium hydroxide are prepared and mixed in a 1:3 wt./wt. ratio referred to as acid base solution (ABS). The ABS is slowly added to the soy protein suspension while stirring at 80-100° C. in a boiling water bath. A 20% wt./wt. aqueous solution of sodium carbonate is prepared and added to the previous mixture and stirred for 7 minutes. A ratio of 1:1 wt./wt. soy glycerin to protein is added slowly. Water evaporated considerably when left to mix in heat for over 2 hours. A film is obtained.

### Example 6

F08-E A suspension comprising 10% wt./wt. soy protein isolate is prepared and sonicated for three minutes. A 20% wt./wt. aqueous solution of maleic acid solution and a 20% wt./wt. aqueous solution of sodium hydroxide are prepared and mixed in a 1:3 wt./wt. ratio referred to as acid base solution (ABS). The ABS is slowly added to the soy protein suspension while stirring at 80-100° C. in a boiling water bath. A 20% wt./wt. aqueous solution of sodium carbonate is prepared and added to the previous mixture and stirred for 7 minutes. A ratio of 1:2 wt./wt. soy glycerin to protein is added slowly. The mixture is dried placing an air hose in the mixture with a continuous flow of air while continuing mixing. A film is formed but no bubbles are successfully formed.

### Example 7

F08-F A suspension comprising 10% wt./wt. soy protein isolate is prepared and sonicated for three minutes. A 20% wt./wt. aqueous solution of maleic acid solution and a 20% wt./wt. aqueous solution of sodium hydroxide are prepared and mixed in a 1:3 wt./wt. ratio referred to as acid base solution (ABS). The ABS is slowly added to the soy protein suspension while stirring at 80-100° C. in a boiling water bath. A 20% wt./wt. aqueous solution of sodium carbonate is prepared and added to the previous mixture and stirred for 7 minutes. A ratio of 1:2 wt./wt. soy glycerin to protein is added slowly. The mixture is dried placing an air hose in the mixture with a continuous flow of air. A film is formed but no bubbles are successfully formed.

### Example 8

F08-G A suspension comprising 10% wt./wt. soy protein isolate is prepared and sonicated for three minutes. A 20% wt./wt. aqueous solution of maleic acid solution and a 20% wt./wt. aqueous solution of sodium hydroxide are prepared and mixed in a 1:3 wt./wt. ratio referred to as acid base solution (ABS). The ABS is slowly added to the soy protein suspension while stirring at 80-100° C. in a boiling water bath for 10 minutes. A ratio of 1:8 wt./wt. of maleic acid to protein is slowly added to the mixture and then sonicated for three minutes. A ratio of 3:8 of soy glycerin to protein is added. Two experiments took place by splitting the mixture into two, adding sodium bicarbonate in one and sodium carbonate on the other one. No significant reaction is observed but it is apparent that sodium bicarbonate works better than sodium carbonate in a 20% wt./wt. aqueous concentration.

### Example 9

F08-H A suspension comprising 10% wt./wt. soy protein isolate is prepared and sonicated for three minutes. A 20% wt./wt. aqueous solution of maleic acid solution and a 20% wt./wt. aqueous solution of sodium hydroxide are prepared and mixed in a 1:3 wt./wt. ratio referred to as acid base solution (ABS). The ABS is slowly added to the soy protein suspension while stirring at 80-100° C. in a boiling water bath for 15 minutes. A ratio of 1:4 wt./wt. of maleic acid to protein is added to the mixture and then sonicated for three minutes. A ratio of 1:2 of soy glycerin to protein is added. Two experiments are preformed by splitting the mixture into two. In the first experiment, an additional ratio of 1:4 wt./wt. of maleic acid to protein and the same ratio of sodium bicarbonate are added to the mixture. This experiment generates a significant amount of bubbling in the film. In the second experiment, additional ratios of 1:8 wt./wt. of maleic acid and 1:4 wt./wt of sodium bicarbonate to protein are added to the mixture. This experiment also generates a significant amount of bubbling in the film but less than the first experiment.

### Example 10

F08-I A suspension comprising 10% wt./wt. soy protein isolate is prepared and sonicated for three minutes. A 20% wt./wt. aqueous solution of maleic acid solution and a 20% wt./wt. aqueous solution of sodium hydroxide are prepared and mixed in a 1:3 wt./wt. ratio referred to as acid base solution (ABS). The ABS is slowly added to the soy protein suspension while stirring at 280 rpm at 80-100° C. in a boiling water bath for 30 minutes. A ratio of 1:2 wt./wt. of soy glycerin to protein is added and mixed at the same rate for 2 minutes under the same heating conditions. A 1:2 wt./wt. ratio of maleic acid to protein is added one hour later and then sonicated for 5 minutes. A 1:2 wt./wt. ratio of sodium bicarbonate to protein is added and mixed. Superficial foam is obtained.

### Example 11

F08-J A suspension comprising 10% wt./wt. soy protein isolate is prepared and sonicated for three minutes. A 20% wt./wt. aqueous solution of maleic acid solution and a 20% wt./wt. aqueous solution of sodium hydroxide are prepared and mixed in a 1:3 wt./wt. ratio referred to as acid base solution (ABS). The ABS is slowly added to the soy protein suspension while stirring at 280 rpm at 80-100° C. in a boiling water bath for 30 minutes. A ratio of 1:2 wt./wt. of soy glycerin to protein is added and mixed at the same rate for 2 minutes under the same heating conditions. A 1:4 wt./wt. ratio of maleic acid to protein is added 30 minutes later and then sonicated for 5 minutes. A 1:2 wt./wt. ratio of sodium bicarbonate to protein is added and mixed. Again, superficial foam is obtained.

### Example 12

F08-P Wax is added to the formulation F08-C in a ratio of about 2:33 wt./wt.

### Example 13

F08-1 An aqueous suspension comprising 10% wt./wt. soy protein isolate is prepared and sonicated for three minutes. This is called F08 formulation. A 20% wt./wt. aqueous solution of maleic acid solution and a 20% wt./wt. aqueous solution of sodium hydroxide are prepared and mixed in a 1:3 wt./wt. ratio referred to as acid base solution (ABS). A formulation called F08-1 is created utilizing 87% F08, 2% ABS, 9% soy glycerin, and 2% wt./wt. maleic acid.

### Example 14

F08-1E A new suspension comprising of 51% F08-1, 3% soy fiber, 11% of a 3% aqueous hydrogen peroxide solution, 17% water, 6% soap, 3% of a 20% aqueous maleic acid solution, and 3% of a 20% aqueous sodium bicarbonate solution is formed under gravimetric conditions using the following method. F08-1 and soy fiber are sonicated together for three minutes. Separately, soap and hydrogen peroxide are mixed together. Parallelly, the water and yeast are combined and warmed at 40° C. for 15 minutes to activate the yeast. Once this step is complete, both the maleic acid and the sodium bicarbonate solutions are added to the main mixture composed of FO8-1 and soy fiber. Then, the soap and hydrogen peroxide solution are also added to this mixture followed by the activated yeast and the ABS. Once formed suspension is divided into two and cooled to i) room temperature once and ii) inside a convective oven set at 40° C. overnight.

### Example 15

F08-1F A new suspension comprising of 51% FO8-1, 3% soy flour, 11% of a 3% aqueous hydrogen peroxide solution, 17% water, 6% soap, 3% of a 20% aqueous maleic acid solution, and 3% of a 20% aqueous sodium bicarbonate solution is formed under gravimetric conditions using the following method. FO8-1 and soy flour are sonicated together for three minutes. Separately, soap and hydrogen peroxide are mixed together. Parallelly, the water and yeast are combined and warmed at 40° C. for 15 minutes to activate the yeast. Once this step is complete, both the maleic acid and the sodium bicarbonate solutions are added to the mixture composed of FO8-1 and soy flour. Then, the soap and hydrogen peroxide solution are also added to this mixture followed by the activated yeast and the ABS. The formed suspension is divided into two and cooled down to i) room temperature and ii) inside a convective oven set at 40° C. overnight.

### Example 16

F08-1G F08-1 is heated and boiled for 5 minutes. Soy glycerin is added followed by a 20% wt/wt. aqueous maleic acid solution. A new suspension is created under gravimetric conditions made up of 95% of the previous mixture and 5% soy flour which is sonicated for three minutes. Then, in a 1:2 wt./wt. ratio, soap and a 15% wt./wt. aqueous hydrogen peroxide solution is created and added to the mixture in a 3:10 wt./wt. ratio. Parallelly, water and yeast are combined in a 3:1 wt./wt. ratio and warmed at 40° C. while mixing at 250 rpm. After activation, yeast-water mixture is added to the previous mixture and cooled to i) room temperature conditions and ii) inside a convective oven set at 40° C.

### Example 17

Soybean cake 1 A suspension is created utilizing a 1:4:40 wt./wt. ratio of the acid base solution (ABS), soy protein isolate, and water. Parallelly, another suspension is created consisting of 62% soy flour, 16% sodium bicarbonate, 3% lecithin, and 19% wt./wt. polyol soybean oil. These two suspensions are combined in a 45:16 ratio, respectively. Yeast is then added and the cake is grown and dried in a vacuum oven.

### Example 18

FM-1 A foam is created comprising 19% of a 30% hydrogen peroxide solution, 10% soap, 16% corn syrup, 16% yeast, and 39% wt./wt. deionized water. First, the yeast is mixed with water and activated in a water bath set at 40° C. for 15 min and stirred at 250 rpm. Parallelly, the hydrogen peroxide solution is mixed with soap and corn syrup and manually stirred. After the yeast is activated, the suspension composed of hydrogen peroxide is added to the former.

### Example 19

FM-1A A foam is created using 56% FM-1, 22% S-155 soybean wax, and 22% wt./wt. polyol soybean oil.

### Example 20

FM-1B A foam is created using 86% FM-1, 7% S-155 soybean wax, and 7% wt./wt. polyol soybean oil.

### Example 21

FM-1C A foam is created using 68% FM-1, 27% S-155 soybean wax, and 5% wt./wt. polyol soybean oil.

### Example 22

FM-1D A foam is created using 68% FM-1, 5% S-155 soybean wax, and 27% wt./wt. polyol soybean oil.

### Example 23

E02-X A 1:10 wt./wt. suspension of soy protein in water is warmed in a water bath set between 80-100° C. and stirred at 220 rpm. After 10 minutes, the ABS solution is added in a 1:44 wt./wt. ratio with respect to the parent suspension and stirred for 10 minutes. Afterwards, soy glycerin is added to the mixture in a 1:11 wt./wt. ratio. After 11 minutes, soy wax S-155 (melted) is added in a 5:4 wt./wt. ratio of wax to soy glycerin. The mixing conditions (rpm and temperature) are kept constant throughout the entire process. Three minutes later, the suspension is sonicated for 3 minutes forming an emulsion which is then placed back in the water bath. Parallelly, a water bath set at 40° C. is prepared to activate a yeast-water suspension in a 26:74% wt./wt. ratio and stirred at 200 rpm for about 20 minutes. Additionally, a hydrogen peroxide solution at 30% is mixed with soap and corn syrup at a 70:24:6% wt./wt. ratio and manually stirred. The yeast-water suspension and hydrogen peroxide solution were then mixed with the parent protein emulsion.

E04 A 1:10 wt./wt. suspension of soy protein in water is warmed in a water bath set between 80-100° C. and stirred at 220 rpm. After 10 minutes, the ABS solution is added in a 1:44 wt./wt. ratio with respect to the parent suspension and stirred for 10 minutes. Afterwards, soy glycerin is added to the mixture in a 2:45 wt./wt. ratio. After 12 minutes, soy wax S-155 (melted) is added in a 5:2 wt./wt. ratio of wax to soy glycerin. The mixing conditions (rpm and temperature) are kept constant throughout the entire process. Three minutes later, the suspension is sonicated for 3 minutes forming an emulsion which is then placed back in the water bath. Parallelly, a water bath set at 40° C. is prepared to activate a yeast-water suspension in a 26:74% wt./wt. ratio and stirred at 200 rpm for 20 minutes. Additionally, a hydrogen peroxide solution at 30% is mixed with soap and corn syrup at a 70:24:6% wt./wt. ratio and manually stirred. The yeast-water suspension and hydrogen peroxide solution are then mixed with the parent protein emulsion.

### Example 25

E05 The formulation for E02 is repeated, using soy wax S-300 instead of soy wax S-155.

### Example 26

E06 The formulation for E04 is repeated, using soy wax S-300 instead of soy wax S-155.

### Example 27

F08-1 Protein crosslinking and structure agents A 1:10 wt./wt. suspension of soy protein in water is warmed up in a water bath set between 80-100° C. and stirred at 220 rpm. After 10 minutes, the ABS solution is added in a 1:44 wt./wt. ratio with respect to the parent suspension and stirred for 10 minutes. Afterwards, soy glycerin is added to the mixture in a 1:11 wt./wt. ratio. After 11 minutes, soy wax S-155 (melted) is added in a 5:4 wt./wt. ratio of wax to soy glycerin. The mixing conditions (rpm and temperature) are kept constant throughout the entire process. Three minutes later, the suspension is sonicated for 3 minutes forming an emulsion which is then returned to the water bath.

### Example 28

FM1 Foaming agents. A water bath set at 40° C. is prepared to activate a yeast-water suspension in a 26:74% wt./wt. ratio and stirred at 200 rpm for 20 minutes (FM1-agent 1). Parallelly, a hydrogen peroxide solution at 30% is mixed with soap and corn syrup at a 70:24:6% wt./wt. ratio and manually stirred (FM1-agent 2). Both products are set in separated beakers ready to be mixed with the parent protein emulsion.

### Example 29

A foaming source that includes the use of PVP. Foaming tests with Polyvinylpyrrolidone (PVP) instead of corn syrup. Four experiments are conducted replacing corn syrup with PVP solutions or a mixture of corn syrup and PVP solution as show in Table 1.

TABLE 1
Run code, experimental keys, reagents used
to test different stablizing agents.

| Run | Code | Reagent |
|---|---|---|
| a. | FM1 | Corn syrup |
| b. | FM1-1 | 0.2% PVP solution |
| c. | FM1-2 | 2% PVP solution |
| d. | FM1-3 | 50:50% wt./wt. corn syrup and best PVP |
| | | solution from b and c |

In these experiments, F08-1 emulsion is formed. Separately, alternative FM1-X solutions are prepared using PVP and/or corn syrup as presented above. FM1-X agent 1 and agent 2 are then simultaneously mixed with F08-1 emulsion. Foam is formed on every case and set inside a convective oven set at 40° C. It is apparent that both PVP and corn syrup provide similar foaming properties in terms of volumetric expansion, texture, and liquid deposition. However, upon drying inside the oven FM1-1 volumetric expansion decreased more than FM1 and FM1-2 after.

### Example 30

Additional foaming tests. The first test studies the foaming effects of sodium bicarbonate. First, soy wax is melted in a water bath at 80-90° C. Then, a 20% wt./wt. aqueous solution of maleic acid and a 20% wt./wt. aqueous solution of sodium bicarbonate are added in a ratio of 5:4:50 wt./wt. maleic acid, sodium bicarbonate, and soy wax. A chemical foaming reaction occurs.

The second test studies the foaming effects of yeast. First, soy wax is melted in a water bath. At the same time, a solution of 7:20 wt./wt. yeast to water is prepared, warmed at 40° C., and stirred at 200 rpm to activate the yeast. This yeast suspension is then added to the wax in a 27:25 wt./wt. ratio, respectively. As this occurs, a 30% hydrogen peroxide solution, soap, and corn syrup are all added by droplets. Eventually a foam forms and is dried in an oven.

### Example 31

E02-4 First a suspension of 1:4:10 wt./wt. ratio of the acid base solution (ABS), soy protein, and water are mixed for ten minutes. Soy glycerin is added in a 2:45 wt./wt. ratio with respect to the previous suspension along with soy wax S-155 in a 1:9 wt./wt. ratio with respect to the previous mixture. A hand mixer with a whisk attachment is used to mix the solution and then it is sonicated for 3 minutes. Afterwards, polyethylene oxide is added while mixing. Polyethylene oxide is added in a ratio of 3:520 wt./wt. with the whole solution. Additionally, the FM1 experiment solution is added using 5% PVP as the independent variable. It is observed that E02 and E02-4 formulations provide the best foaming conditions. However, larger amounts of soy glycerin in E02-4 may be needed to optimize foam formed in this method.

### Example 32

FM1-1A A water bath set at 40° C. is prepared to activate a yeast-water suspension in a 26:74% wt./wt. ratio and stirred at 200 rpm for 20 minutes (FM1-1A-agent 1). Parallelly, a hydrogen peroxide solution at 30% is mixed with soap and corn syrup at a 70:24:6% wt./wt. ratio and manually stirred (FM1-1A-agent 2). Both products are set in separated beakers ready to be mixed with the parent protein emulsion.

### Example 33

FM1-1B A water bath set at 40° C. is prepared to activate a yeast-water suspension in a 26:74% wt./wt. ratio and magnetically stirred at 200 rpm for 20 minutes (FM1-1B-agent 1). Parallelly, a hydrogen peroxide solution at 30% is mixed with soap and PVP (5%) at a 70:24:6% wt./wt. ratio and manually stirred (FM1-1B-agent 2). Both products are set in separated beakers ready to be mixed with the parent protein emulsion.

### Example 34

F08-1A A 1:5 wt./wt. suspension of soy protein in water is warmed in a water bath set between 80-100° C. and stirred at 220 rpm. The ABS solution is added in a 1:48 wt./wt. ratio with respect to the parent suspension and stirred for 10 minutes. Afterwards, soy glycerin is added to the mixture in a 4:49 wt./wt. ratio. After 12 minutes, soy wax S-155 (melted) is added in a 5:4 wt./wt. ratio of wax to soy glycerin. The mixing conditions (rpm and temperature) are kept constant throughout the entire process. Three minutes later, the suspension is sonicated for 3 minutes forming an emulsion which is then placed back in the water bath.

### Example 35

F08-1B A 1:10 wt./wt. suspension of soy protein in water is warmed in a water bath set between 80-100° C. and stirred at 220 rpm. The ABS solution is added in a 1:44 wt./wt. ratio with respect to the parent suspension and stirred for 10 minutes. Afterwards, soy glycerin is added to the mixture in a 4:45 wt./wt. ratio. After 12 minutes, soy wax S-155 (melted) is added in a 5:2 wt./wt. ratio of wax to soy glycerin. The mixing conditions (rpm and temperature) are kept constant throughout the entire process. Three minutes later, the suspension is sonicated for 3 minutes forming an emulsion which is then placed back in the water bath.

### Example 36

E02-F1 Referring to the mixture between F08-1B and FM1-1A. Using F08-1B as a base emulsion, FM1-1A-agent 1 is added and mixed using a hand mixer with a whisk attachment. Then, FM1-1A-agent 2 is poured into the mixture. Foam is formed and dried in a convection oven set at 40° C. overnight.

### Example 37

E02-F2 Referring to the mixture between F08-1A and FM1-1A. Using F08-1A as a base emulsion, FM1-1A-agent 1 is added and mixed using a hand mixer with a whisk attachment. Then, FM1-1A-agent 2 is poured into the mixture. Foam is formed and dried in a convection oven set at 40° C. overnight.

### Example 38

E02-4-F1 Referring to the mixture between F08-1B and FM1-1B. Using F08-1B as a base emulsion, FM1-1B-agent 1 is added and mixed using a hand mixer with a whisk attachment. Then, FM1-1B-agent 2 is poured into the mixture. Foam is formed and dried in a convection oven set at 40° C. overnight.

### Example 39

E02-4-F2 Referring to the mixture between F08-1A and FM1-1B. Using F08-1A as a base emulsion, FM1-1B-agent 1 is added and mixed using a hand mixer with a whisk attachment. Then, FM1-1B-agent 2 is poured into the mixture. Foam is formed and dried in a convection oven set at 40° C. overnight.

### Example 40

E02 A 1:10 wt./wt. suspension of soy protein in water is warmed in a water bath set between 80-100° C. and stirred at 220 rpm. The ABS solution is added in a 1:44 wt./wt. ratio with respect to the parent suspension and stirred for 10 minutes. Afterwards, soy glycerin is added to the mixture in a 4:45 wt./wt. ratio. After 18 minutes, soy wax S-155 (melted) is added in a 5:4 wt./wt. ratio of wax to soy glycerin. The mixing conditions (rpm and temperature) are kept constant throughout the entire process. Three minutes later, the suspension is sonicated for 3 minutes forming an emulsion which is then placed back in the water bath for about 30-50 minutes to allow it to cool to about 30-50° C.

### Example 41

FM1-1C A water bath set at 40° C. is prepared to activate a yeast-water suspension in a 23:77% wt./wt. ratio and stirred at 200 rpm for 20 minutes (FM1-1C-agent 1). Parallelly, a hydrogen peroxide solution at 30% is mixed with soap and corn syrup at a 70:24:6% wt./wt. ratio and manually stirred (FM1-1C-agent 2). Both products were set in separated beakers ready to be mixed with the parent protein emulsion.

### Example 42

FM1-1D A water bath set at 40° C. is prepared to activate a yeast-water suspension in a 23:77% wt./wt. ratio and stirred at 200 rpm for 20 minutes (FM1-1D-agent 1). Parallelly, a hydrogen peroxide solution at 30% is mixed with soap and PVP (5%) at a 70:24:6% wt./wt. ratio and manually stirred (FM1-1D-agent 2). Both products are set in separated beakers ready to be mixed with the parent protein emulsion.

### Example 43

S01 Referring to the mixture between E02 and FM1-1B. Using E02 as a base emulsion, FM1-1B-agent 1 is added and mixed using a hand mixer with a whisk attachment. Then, FM1-1B-agent 2 is poured into the mixture. Foam is formed and dried in a convection oven set at 40° C. overnight.

### Example 44

S02 Referring to the mixture between E02 and FM1-1A. Using E02 as a base emulsion, FM1-1A-agent 1 is added and mixed using a hand mixer with a whisk attachment. Then, FM1-1A-agent 2 is poured into the mixture. Foam is formed and dried in a convection oven set at 40° C. overnight.

### Example 44

S03 Referring to the mixture between E02 and FM1-1D. Using E02 as a base emulsion, FM1-1D-agent 1 is added and mixed using a hand mixer with a whisk attachment. Then, FM1-1D-agent 2 is poured into the mixture. Foam is formed and dried in a convection oven set at 40° C. overnight.

### Example 45

S04 Referring to the mixture between E02 and FM1-1C. Using E02 as a base emulsion, FM1-1C-agent 1 is added and mixed using a hand mixer with a whisk attachment. Then, FM1-1C-agent 2 is poured into the mixture. Foam is formed and dried in a convection oven set at 40° C. overnight.

### Example 46

P01: A 1:10 wt./wt. suspension of hemp protein in water is warmed in a water bath set between 80-100° C. and stirred at 220 rpm. The ABS solution is added in a 1:44 wt./wt. ratio with respect to the parent suspension and stirred for 10 minutes. Afterwards, soy glycerin is added to the mixture in a 4:45 wt./wt. ratio. After 18 minutes, soy wax S-155 (melted) is added in a 5:4 wt./wt. ratio of wax to soy glycerin. The mixing conditions (rpm and temperature) are kept constant throughout the entire process. Three minutes later, the suspension is sonicated for 3 minutes forming an emulsion which is then placed back in the water bath for about 30-50 minutes to allow it cool to about 30-50° C. Subsequently, using P01 as a base emulsion, FM1-1A-agent 1 is added and mixed using a hand mixer with a whisk attachment. Then, FM1-1A-agent 2 is poured into the mixture. Foam is formed and dried in a convection oven set at 40° C. for 3-5 h.

### Example 47

P02: A 1:10 wt./wt. suspension of pea protein in water is warmed in a water bath set between 80-100° C. and stirred at 220 rpm. The ABS solution is added in a 1:44 wt./wt. ratio with respect to the parent suspension and stirred for 10 minutes. Afterwards, soy glycerin is added to the mixture in a 4:45 wt./wt. ratio. After 18 minutes, soy wax S-155 (melted) is added in a 5:4 wt./wt. ratio of wax to soy glycerin. The mixing conditions (rpm and temperature) are kept constant throughout the entire process. Three minutes later, the suspension is sonicated for 3 minutes forming an emulsion which is then placed back in the water bath for about 30-50 minutes to allow it to cool to about 30-50° C. Subsequently, using P02 as a base emulsion, FM1-1A-agent 1 is added and mixed using a hand mixer with a whisk attachment. Then, FM1-1A-agent 2 is poured into the mixture. Foam is formed and dried in a convection oven set at 40° C. for 3-5 h or overnight.

### Example 48

P03: A 1:10 wt./wt. suspension of casein protein in water is warmed in a water bath set between 80-100° C. and stirred at 220 rpm. The ABS solution is added in a 1:44 wt./wt. ratio with respect to the parent suspension and stirred for 10 minutes. Afterwards, soy glycerin is added to the mixture in a 4:45 wt./wt. ratio. After 18 minutes, soy wax S-155 (melted) is added in a 5:4 wt./wt. ratio of wax to soy glycerin. The mixing conditions (rpm and temperature) are kept constant throughout the entire process. Three minutes later, the suspension is sonicated for 3 minutes forming an emulsion which is then returned to the water bath for about 30-50 minutes to then let it cool to about 30-50° C. Subsequently, using P03 as a base emulsion, FM1-1A-agent 1 is added and mixed using a hand mixer with a whisk attachment. Then, FM1-1A-agent 2 is poured into the mixture. Foam is formed and dried in a convection oven set at 40° C. for 3-5 h or overnight.

### Example 49

Some additional proteins useful for creating foams. Experiments encompassing the following proteins will result in good looking foams with the potential to be used for packaging purposes.

TABLE 2
Experimental keys and some of the proteins
useful for the creation of foams.

| Key | Protein |
|---|---|
| P04 | comprising flaxseed protein isolate |
| P05 | comprising whey protein isolate |
| P06 | comprising rice protein powder |
| P07 | comprising zein protein |
| P08 | comprising egg white protein isolate |
| P09 | comprising fish protein isolate |
| P01 | comprising hemp |
| P02 | comprising pea protein |
| P03 | containig casein protein |

For P04, P05, P06, P07, P08, P09. A 1:10 wt./wt. suspension of P04, P05, P06, P07, P08, OR P09 in water are warmed in a water bath set between 80-100° C. and stirred at 220 rpm. The ABS solution is then added as a 1:44 wt./wt. ratio with respect to the parent suspension and is stirred for 10 minutes. Afterwards, soy glycerin is added to the mixture in a 4:45 wt./wt. ratio. After 18 minutes, soy wax S-155 (melted) is added in a 5:4 wt./wt. ratio of wax to soy glycerin. The mixing conditions (rpm and temperature) are kept constant throughout the process. Three minutes later, the suspension is sonicated for 3 minutes forming an emulsion which is then returned to the water bath for about 30-50 minutes to then let it cool to about 30-50° C. Subsequently, using P04, P05, P06, P07, P08, or P09 as the base emulsion, FM1-1A-agent 1 is added and mixed using a hand mixer with a whisk attachment. Then, FM1-1A-agent 2 is poured into the mixture. Foam is formed and dried in a convection oven set at 40° C. for 3-5 h.

### Example 50

W06: A 1:10 wt./wt. suspension of soy protein isolate in water is warmed in a water bath set between 80-100° C. and stirred at 220 rpm. The ABS solution is added in a 1:44 wt./wt. ratio with respect to the parent suspension and stirred for 10 minutes. Afterwards, soy glycerin is added to the mixture in a 4:45 wt./wt. ratio. After 18 minutes, beeswax (melted) is added in a 5:4 wt./wt. ratio of wax to soy glycerin. The mixing conditions (rpm and temperature) are kept constant throughout the process. Three minutes later, the suspension is sonicated for 3 min03utes forming an emulsion which is then placed back in the water bath for about 30-50 minutes to then let it cool to about 30-50° C. Using W06 as a base emulsion, FM1-1A-agent 1 is added and mixed using a hand mixer with a whisk attachment. Then, FM1-1A-agent 2 is poured into the mixture. Foam is formed and dried in a convection oven set at 40° C. for 3-5 h.

### Example 51

W07: A 1:10 wt./wt. suspension of soy protein in water is warmed in a water bath set between 80-100° C. and stirred at 220 rpm. The ABS solution is added in a 1:44 wt./wt. ratio with respect to the parent suspension and stirred for 10 minutes. Afterwards, soy glycerin is added to the mixture in a 4:45 wt./wt. ratio. After 18 minutes, carnauba (melted) is added in a 5:4 wt./wt. ratio of wax to soy glycerin. The mixing conditions (rpm and temperature) were kept constant throughout the entire process. Three minutes later, the suspension is sonicated for 3 minutes forming an emulsion which is then returned to the water bath for about 30-50 minutes to then let it cool to about 30-50° C. Using W07 as a base emulsion, FM1-1A-agent 1 is added and mixed using a hand mixer with a whisk attachment. Then, FM1-1A-agent 2 is poured into the mixture. Foam is formed and dried in a convection oven set at 40° C. for 3-5 h.

### Example 52

W08: A 1:10 wt./wt. suspension of soy protein in water is warmed in a water bath set between 80-100° C. and stirred at 220 rpm. The ABS solution is added in a 1:44 wt./wt. ratio with respect to the parent suspension and is stirred for 10 minutes. Afterwards, soy glycerin is added to the mixture in a 4:45 wt./wt. ratio. After 18 minutes, paraffin wax (melted) is added in a 5:4 wt./wt. ratio of wax to soy glycerin. The mixing conditions (rpm and temperature) are kept constant throughout the process. Three minutes later, the suspension is sonicated for 3 minutes forming an emulsion which is then placed back in the water bath for about 30-50 minutes to then let it cool to about 30-50° C. Using W08 as a base emulsion, FM1-1A-agent 1 is added and mixed using a hand mixer with a whisk attachment. Then, FM1-1A-agent 2 is poured into the mixture. Foam is formed and dried in a convection oven set at 40° C. for 3-5 h.

### Example 53

TABLE 2
List of waxes useful for the manufacture
of some of the inventive foams

| Code | Type of Wax |
|---|---|
| W09 | comprising candelilla wax |
| W10 | comprising coconut wax |
| W11 | comprising casting wax |
| W12 | comprising microcrystalline wax |
| W06 | comprising beeswax |
| W07 | comprising carnauba |
| W08 | comprising paraffin |

For W09, W10, W11, and W12. A 1:10 wt./wt. suspension of soy protein in water is warmed in a water bath set between 80-100° C. and stirred at 220 rpm. The ABS solution is added as a 1:44 wt./wt. ratio with respect to the parent suspension and is stirred for 10 minutes. Afterwards, soy glycerin is added to the mixture in a 4:45 wt./wt. ratio. After 18 minutes, wax W09, W10, W11, or W12 (melted) are added in a 5:4 wt./wt. ratio of wax to soy glycerin. The mixing conditions (rpm and temperature) are kept constant throughout the process. Three minutes later, the suspension is sonicated for 3 minutes forming an emulsion which is then returned to the water bath for about 30-50 minutes to then let it cool to about 30-50° C. Using W09, W10, W11, or W12 as a base emulsion, FM1-1A-agent 1 is added and mixed using a hand mixer with a whisk attachment. Then, FM1-1A-agent 2 is poured into the mixture. Foam is formed and dried in a convection oven set at 40° C. for 3-5 h.

### Example 54

TABLE 3
A list of some acids and bases that may useful
for the manufacture of biodegradable foams.

| Code | Types of Acids |
|---|---|
| A01 | including malic acid |
| A02 | including aspartic acid |
| A03 | including adipic acid dihydrazide |
| A04 | including adipic acid |
| A05 | including glutamic acid |
| A06 | including citric acid |
| A07 | including tartaric acid |
| A08 | including oxalic acid |
| A09 | including azelaic acid |
| A10 | including fumaric acid |

For A01, A02, A03, A04, A05, A06, A07, A08, A09, and A10. A 1:10 wt./wt. suspension of soy protein in water is warmed in a water bath set between 80-100° C. and stirred at 220 rpm. The ABS solution comprising A01, A02, A03, A04, A05, A06, A07, A08, A09, or A10 is then added as a 1:44 wt./wt. ratio with respect to the parent suspension and is stirred for 10 minutes. Afterwards, soy glycerin is added to the mixture in a 4:45 wt./wt. ratio. After 18 minutes, soy wax (melted) is added in a 5:4 wt./wt. ratio of wax to soy glycerin. The mixing conditions (rpm and temperature) are kept constant throughout the entire process. Three minutes later, the suspension is sonicated for 3 minutes forming an emulsion which is then returned to the water bath for about 30-50 minutes to then let it cool to about 30-50° C. Using A01, A02, A03, A04, A05, A06, A07, A08, A09, or A10 as a base emulsion, FM1-1A-agent 1 is added and mixed using a hand mixer with a whisk attachment. Then, FM1-1A-agent 2 is poured into the mixture. Foam is formed and dried in a convection oven set at 40° C. for 3-5 h.

Bases: B00: The corresponding bases that neutralize the above-mentioned dicarboxylic acids.

For B00: A 1:10 wt./wt. suspension of soy protein in water is warmed in a water bath set between 80-100° C. and stirred at 220 rpm. The ABS solution comprising B00 is added as a 1:44 wt./wt. ratio with respect to the parent suspension and is stirred for 10 minutes. Afterwards, soy glycerin is added to the mixture in a 4:45 wt./wt. ratio. After 18 minutes, soy wax (melted) is added in a 5:4 wt./wt. ratio of wax to soy glycerin. The mixing conditions (rpm and temperature) is kept constant throughout the entire process. Three minutes later, the suspension is sonicated for 3 minutes forming an emulsion which is then placed back in the water bath for about 30-50 minutes to then let it cool to about 30-50° C. Using B00 as a base emulsion, FM1-1A-agent 1 is added and mixed using a hand mixer with a whisk attachment. Then, FM1-1A-agent 2 is poured into the mixture. Foam is formed and dried in a convection oven set at 40° C. for 3-5 h.

### Example 55

A biodegradable moldable soy based foam, is formed as follows. A parent suspension comprising a 1:5 wt./wt. suspension of soy protein in water, warmed up in a water bath set between 80-100° C. and stirred at 220 rpm. Next a ABS solution, comprising a 20% wt./wt. aqueous solution of maleic acid solution and a 20% wt./wt. aqueous solution of sodium hydroxide are prepared and mixed in a 1:3 wt./wt. ratio referred to as acid base solution (ABS is added in a 1:48 wt./wt. ratio with respect to the parent suspension and stirred for 10 minutes. Afterwards, soy glycerin is added to the mixture in a 4:49 wt./wt. ratio. After 12 minutes, soy wax S-155 (melted) is added in a 5:4 wt./wt. ratio of wax to soy glycerin. The mixing conditions (rpm and temperature) are kept constant throughout the entire process. Three minutes later, the suspension is sonicated for 3 minutes forming an emulsion which was then placed back in the water bath for about 30-50 minutes to then let it cool to about 30-50° C.

Concurrently, a water bath set at 40° C. was prepared to activate a yeast-water suspension in a 26:74% wt./wt. ratio and magnetically stirred at 200 rpm for 20 minutes (FM1-1A-agent 1). Parallelly, a hydrogen peroxide solution at 30% was mixed with soap and corn syrup at a 70:24:6% wt./wt. ratio and manually stirred (FM1-1A-agent 2). Both products were set in separated beakers ready to be mixed with the parent protein emulsion.

FM1-1A-agent 1 is added to the emulsion with mixing. Next, FM1-1-agent 2 is poured into the mixture of the emulsion and FM1-1A-agent 1, to form a foam. The foam is dried for 3-6 h or overnight in a convection oven set at 40° C.

### Example 56

A biodegradable moldable soy based foam is formed as follows. A 1:10 wt./wt. suspension of soy protein in water is warmed up in a water bath set between 80-100° C. and magnetically stirred at 220 rpm. After 10 minutes, an ABS solution comprising a 20% wt./wt. aqueous solution of maleic acid solution and a 20% wt./wt. aqueous solution of sodium hydroxide are prepared and mixed in a 1:3 wt./wt. ratio referred to as acid base solution (ABS is added in a 1:44 wt./wt. ratio with respect to the parent suspension and stirred for 10 minutes.

Afterwards, soy glycerin is added to the mixture in a 1:11 wt./wt. ratio. After 11 minutes, soy wax S-155 (melted) is added in a 5:4 wt./wt. ratio of wax to soy glycerin. The mixing conditions (rpm and temperature) are kept constant throughout the entire process. Three minutes later, the suspension is sonicated for 3 minutes forming an emulsion which is then placed back in the water bath for about 30-50 minutes to then let it cool to about 30-50° C. Parallelly, a water bath set at 40° C. is prepared to activate a yeast-water suspension in a 26:74% wt./wt. ratio and stirred at 200 rpm for 20 minutes. Additionally, a hydrogen peroxide solution at 30% is mixed with soap and corn syrup at a 70:24:6% wt./wt. ratio and manually stirred. The yeast-water suspension and hydrogen peroxide solution are then mixed with the parent protein emulsion to form a base emulsion.

Concurrently, a water bath set at 40° C. is prepared to activate a yeast-water suspension in a 26:74% wt./wt. ratio and stirred at 200 rpm for 20 minutes (FM1-1A-agent 1). Parallelly, a hydrogen peroxide solution at 30% is mixed with soap and corn syrup at a 70:24:6% wt./wt. ratio and manually stirred (FM1-1A-agent 2). Both products are set in separated beakers ready to be mixed with the parent protein emulsion.

FM1-1A-agent 1 is added to the emulsion and mixed. Then, FM1-1A-agent 2 is added to the emulsion mixed with FM1-1A-agent 1 to form a foam. Finally, the foam is dried for 3-6 hours or overnight is a convection oven set at 40° C.

While this invention has been described as having an exemplary design, the present invention may be further modified within the spirit and scope of this disclosure. This application is therefore intended to cover any variations, uses, or adaptations of the invention using its general principles.

1. A biodegradable foam, comprising:
a cross-linked protein;
a gas;
inactivated yeast; and
a meltable wax;
wherein, the protein forms a matrix that comprises one or more spaces that entraps the gas and the matrix interacts with the wax.

2. The foam according to claim 1, further comprising a plasticizer.

3. The foam according to claim 1, wherein the foam further comprises an excipient.

4. The foam according to claim 3, wherein the excipient is selected from the group consisting of: sugar, polyvinylpyrrolidone, a fiber, and at least one emulsifier.

5. The foam according to claim 4, wherein the emulsifier is selected from the group consisting of: soap and lecithin.

6. The foam according to claim 1, wherein the protein is isolated from soy.

7. The foam according to claim 1, wherein the gas is oxygen, and the oxygen is provided by a reaction between yeast and hydrogen peroxide during the process of forming the foam.

8. The foam according to claim 1, wherein the wax is selected from the group consisting of: soy wax, paraffin, beeswax, candelilla wax, coconut wax, casting wax, microcrystalline wax, carnauba.

9. The foam according to claim 2, wherein the plasticizer is glycerin.

10. The foam according to claim 1, wherein the foam comprises:
about 6 to 10% wt./wt. protein;
about 0.3 to 0.4% wt./wt. of dicarboxylic acid;
about 1.0 to 1.3% wt./wt. of a strong base;
about 3 to 7% wt./wt. glycerol;
about 3 to 7% wt./wt. of a wax;
about 1.5 to 10% wt./wt. of hydrogen peroxide;
about 4.5 to 6.5% wt./wt. of soap; and
about 5 to 7% wt./wt. of yeast.

11. The foam according to claim 1, wherein the foam has a density of between about 60 kg/m³ to about 400 kg/m³.

12. The foam according to claim 1, wherein the foam has a density of between about 78 kg/m³ to about 335 kg/m³.

13. A method of making a biodegradable foam, comprising:
a. adding, with agitation, a crosslinking reagent to an aqueous suspension of a protein held at a temperature of between 80° C. and 100° C. form a mixture;
b. introducing, with stirring, a plasticizer into the mixture;
c. supplying the mixture with a melted meltable wax to create a formulation;
d. providing a source of a gas to the formulation to form a liquid foam; and
e. drying the liquid foam to form a stable foam.

14. The method according to claim 13, further comprising:
emulsifying the formation to create an emulsion before providing the gas to the emulsion.

15. The method according to claim 13, wherein the source of gas is provided by:
adding an activated yeast suspended in water; and a solution of hydrogen peroxide, corn syrup and soap to the emulsion.

16. The method according to claim 13, wherein the protein is selected from the group consisting of: soybeans, flaxseed, whey, rice powder, zein, egg white, casein and fish.

17. The method according to claim 13, wherein the mixture comprises a flour.

18. The method according to claim 13, wherein the protein is a soy isolate.

19. The method according to claim 13, wherein the crosslinking reagent comprises a dicarboxylic acid and a base.

20. The method according to claim 13, furthering comprising:
transferring the liquid foam to a mold; and
drying the liquid foam to form a dried foam.
